(19) United States
(12) Patent Application Publication
(10) Pub. No.: US 2025/0206769 A1
(43) Pub. Date: Jun. 26, 2025

(54) Synthesizing a Nucleic Acid or Nucleic Acid Analogue

(71) Applicant: Imec vzw, Leuven (BE)

(72) Inventor: Kathrin Hoelz, Leuven (BE)

(21) Appl. No.: 18/982,920

(22) Filed: Dec. 16, 2024

(30) Foreign Application Priority Data
Dec. 22, 2023 (EP) 23220036.0

(51) Int. Cl. C07H 21/00; B01J 19/00

(52) U.S. Cl. CPC C07H 21/00; B01J 19/0046; B01J 2219/00596; B01J 2219/00605; B01J 2219/00722

(57) ABSTRACT

In a first aspect, the present disclosure relates to a method for synthesizing a nucleic acid or nucleic acid analogue, comprising: a) providing a root fragment of the nucleic acid or nucleic acid analogue, b) providing a further fragment of the nucleic acid or nucleic acid analogue, c) phosphitylating either the root fragment or the further fragment, and d) binding the further fragment to the root fragment by a phosphoramidite coupling; wherein the root fragment and the further fragment each have a length of at least 5 monomers.

### CROSS-REFERENCE TO RELATED APPLICATIONS

The present application is a non-provisional patent application claiming priority to European Patent Application No. EP 23220036.0, filed Dec. 22, 2023, the contents of which are hereby incorporated by reference.

### FIELD OF THE DISCLOSURE

The present disclosure relates to synthesizing a nucleic acid or nucleic acid analogue, and in particular wherein the synthesis involves phosphoramidite coupling.

### BACKGROUND

Phosphoramidite chemistry is widely used to chemically synthesize (oligo) nucleic acids such as DNA, RNA, other nucleic acids and their analogues. In this workflow, a new building block is added to the surface-tethered growing strand in a monomer addition cycle which exists of four essential steps: coupling, capping, oxidation and detritylation.

In the coupling step, a covalent bond is formed between the incoming phosphoramidite and the immobilized nucleic acid strand. Since the stepwise coupling efficiency of a monomer addition is below 100%, a certain percentage of strands remains unreacted. To prevent those unreacted strands from growing further and resulting in incorrect sequences, they are capped. This allows the separation of full-length products from truncated fragments.

However, due to the finite and compounding stepwise coupling yield, the length of the oligonucleotides that can be achieved using conventional phosphoramidite chemistry is limited, with a length of roughly up to 100-mers for DNA and 60-mers for RNA. With highly optimized protocols, oligonucleotide lengths of up to 200-mers for DNA and up to 120-mers for RNA can sometimes be reached but even these remain fairly short. Nonetheless, the area of applications for various oligonucleotides is growing rapidly; especially since the development of mRNA-based vaccines, which entail a high demand for RNA oligonucleotides of significantly longer length. The current workflow for producing oligonucleotides of such lengths is very labour-intensive and can easily take up several weeks.

US2021047361A1 sought to overcome these challenges and disclosed a method in which-rather than capping unreacted strands and prevent them from growing further-all the strands bear a 5′-OH-thermally cleavable protecting group which can be selectively removed by heating selected sites of the substrate. Upon selective thermal deprotection, a nucleoside 3′-phosphoramidite or a di- or tri-nucleotide 3′-phosphoramidite-again bearing a 5′-OH protecting group—is then coupled onto the deprotected 5′—OH groups. However, this approach has its own inherent limitations and shortcomings.

There is thus still a need in the art for approaches to nucleic acid synthesis which address at least some of the issues outlined above.

### SUMMARY

In a first aspect, the present disclosure provides a method for synthesizing a nucleic acid or nucleic acid analogue. The method includes a) providing a root fragment of the nucleic acid or nucleic acid analogue; b) providing a further fragment of the nucleic acid or nucleic acid analogue; c) phosphitylating either the root fragment or the further fragment; and d) binding the further fragment to the root fragment by a phosphoramidite coupling. The root fragment and the further fragment each have a length of at least 5 monomers.

In one embodiment of the first aspect, a ratio of the length of the further fragment to the length of the root fragment is from 0.8 to 1.2. In one embodiment of the first aspect, in step d the root fragment and the further fragment each have an end rendered unreactive to phosphoramidite coupling.

In one embodiment of the first aspect, in step d the root fragment is attached to a substrate.

In one embodiment of the first aspect, the root fragment and/or the further fragment are synthesized on a substrate.

In one embodiment of the first aspect, the root fragment and/or the further fragment is attached at one end to the substrate by a linker, the linker being such that the fragment can be detached from the substrate while leaving a protective group on the end rendering the end unreactive to phosphoramidite coupling.

In one embodiment of the first aspect, the root fragment and/or the further fragment are synthesized using a method comprising capping an erroneous fragment to prevent further chain elongation, and wherein providing the root fragment and the further fragment with the end rendered unreactive to phosphoramidite coupling is performed such that erroneous fragments are unreactive to phosphoramidite coupling at both ends.

In one embodiment of the first aspect, the root fragment and/or the further fragment are synthesized using phosphoramidite synthesis.

In one embodiment of the first aspect, in step c the root fragment or further fragment is phosphitylated while attached to a substrate.

In one embodiment of the first aspect, the root fragment or further fragment is phosphitylated using chloro N,N,N′,N′-tetraisopropyl phosphorodiamidite.

In one embodiment of the first aspect, after step d the root fragment extended with the further fragment becomes a new root fragment or a new further fragment, and wherein steps a to d are repeated at least 1 time.

In one embodiment of the first aspect, the root fragment and the further fragment each have a 5′ and a 3′ end, and in step d, the root fragment is attached to an assembly substrate through its 5′ end and the further fragment comprises a protective group at its 3′ end, or the root fragment is attached to an assembly substrate through its 3′ end and the further fragment comprises a protective group at its 5′ end. In one embodiment, the root fragment is phosphitylated while attached to the assembly substrate. In one embodiment, the further fragment is synthesized on a synthesis substrate in a 3′ to 5′ direction if the root fragment is in step d attached to the assembly substrate through its 5′ end, or in a 5′ to 3′ direction if the root fragment is in step d attached to the assembly substrate through its 3′ end.

In a second aspect, the present disclosure provides a device for synthesizing a nucleic acid or nucleic acid analogue using the method of the first aspect. The device includes a) a component for receiving the root fragment and the further fragment of the nucleic acid or nucleic acid analogue; b) a component for phosphitylating either the root fragment or the further fragment; and c) a component for binding the further fragment to the root fragment by a phosphoramidite coupling.

### BRIEF DESCRIPTION OF THE FIGURES

The disclosure above, as well as additional features, will be better understood through the following illustrative and non-limiting detailed description of example embodiments, with reference to the drawings.

FIG. 1 schematically depicts a reaction step according to one embodiment of the present disclosure;

FIG. 2 schematically depicts a reaction step according to one embodiment of the present disclosure;

FIG. 3 schematically depicts a reaction step according to one embodiment of the present disclosure;

FIG. 4 schematically depicts a reaction step according to one embodiment of the present disclosure;

FIG. 5 schematically depicts a reaction step according to one embodiment of the present disclosure;

FIG. 6 schematically depicts a reaction step according to one embodiment of the present disclosure;

FIG. 7 schematically depicts a reaction step according to one embodiment of the present disclosure;

FIG. 8 schematically depicts a reaction step according to one embodiment of the present disclosure;

FIG. 9 schematically depicts a reaction step according to one embodiment of the present disclosure; and

FIG. 10 schematically depicts a device for carrying out the methods of the present disclosure.

All the figures are schematic, not necessarily to scale, and generally only show parts which are necessary to elucidate example embodiments, wherein other parts may be omitted or merely suggested.

### DETAILED DESCRIPTION

The present disclosure will be described with respect to particular embodiments and with reference to certain drawings but the disclosure is not limited thereto. The drawings described are schematic and non-limiting. In the drawings, the size of some of the elements may be exaggerated and not drawn on scale for illustrative purposes.

Furthermore, the terms first, second, third and the like in the description and in the claims, are used for distinguishing between similar elements and not necessarily for describing a sequence, either temporally, spatially, in ranking or in any other manner. It is to be understood that the terms so used are interchangeable under appropriate circumstances and that the embodiments of the disclosure described herein are capable of operation in other sequences than described or illustrated herein.

Moreover, the terms top, bottom, over, under and the like in the description and the claims are used for descriptive purposes and not necessarily for describing relative positions. It is to be understood that the terms so used are interchangeable with their antonyms under appropriate circumstances and that the embodiments of the disclosure described herein are capable of operation in other orientations than described or illustrated herein.

The term “comprising”, used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof. The term “comprising” therefore covers the situation where only the stated features are present and the situation where these features and one or more other features are present. Thus, the scope of the expression “a device comprising means A and B” should not be interpreted as being limited to devices consisting only of components A and B. It means that with respect to the present disclosure, some relevant components of the device include A and B.

Similarly, it is to be noticed that the term “coupled”, also used in the claims, should not be interpreted as being restricted to direct connections only. The terms “coupled” and “connected,” along with their derivatives, may be used, but are not necessarily intended as synonyms for each other. Thus, for example, the scope of the expression “a device A coupled to a device B” should not be limited to devices or systems wherein an output of device A is directly connected to an input of device B. It means that there exists a path between an output of A and an input of B which may be a path including other devices or means. In some embodiments, “coupled” can mean that two or more elements are either in direct physical or electrical contact, or that two or more elements are not in direct contact with each other but yet can still co-operate or interact with each other.

Reference throughout this specification to “one embodiment” or “an embodiment” means that a particular feature, structure, or characteristic described in connection with the embodiment is included in at least one embodiment of the present disclosure. Thus, appearances of the phrases “in one embodiment” or “in an embodiment” in various places throughout this specification are not necessarily all referring to the same embodiment, but may. Furthermore, particular features, structures, or characteristics may be combined in any suitable manner, as would be apparent to one of ordinary skill in the art from this disclosure, in one or more embodiments.

Similarly, it should be appreciated that in the description of exemplary embodiments of the disclosure, various features of the disclosure are sometimes grouped together in a single embodiment, figure, or description thereof for the purpose of streamlining the disclosure and aiding in the understanding of one or more of the various aspects. This method of disclosure, however, is not to be interpreted as reflecting an intention that the claimed disclosure requires more features than are expressly recited in each claim. Rather, as the following claims reflect, certain aspects lie in less than all features of a single foregoing disclosed embodiment. Thus, the claims following the detailed description are hereby expressly incorporated into this detailed description, with each claim standing on its own as a separate embodiment of this disclosure.

Furthermore, while some embodiments described herein include some but not other features included in other embodiments, combinations of features of different embodiments are meant to be within the scope of the disclosure, and form different embodiments, as would be understood by those in the art. For example, in the following claims, any of the claimed embodiments can be used in any combination.

In the description provided herein, numerous specific details are set forth. However, it is understood that some embodiments of the disclosure may be practiced without these specific details. In other instances, well-known methods, structures and techniques have not been shown in detail in order to not obscure an understanding of this description.

The following terms are provided solely to aid in the understanding of the disclosure.

As used herein, and unless otherwise specified, “nucleic acid (analogue)” refers to a nucleic acid or a nucleic acid analogue. As known in the art, a “nucleic acid analogue” is a structural analogue to (naturally occurring) nucleic acids, such as DNA (deoxyribonucleic acid) or RNA (ribonucleic acid). Where nucleic acids are chains of nucleotides, which are composed of three parts: a phosphate backbone, pentose sugar (deoxyribose or ribose), and nucleobase (adenine, cytosine, guanine, thymine or uracil); a nucleic acid analogue is a chain of nucleotide analogues, which may have any of those parts altered. In some examples, nucleic acid analogues have a different sugar—also referred to as ‘xeno nucleic acids’ (XNA)—, and/or have modified or artificial nucleobases, and/or have modified backbones.

As used herein, and unless otherwise specified, when an end of a fragment is said to be rendered “less reactive” to phosphoramidite coupling, it is meant that the end is less reactive to phosphoramidite coupling than if the end would bear an —OH group. Similarly, when the end of a fragment is said to be rendered “unreactive” to phosphoramidite coupling, it is meant that the reaction speed of phosphoramidite coupling is at least 10 times lower than if the end would bear an —OH group. In some embodiments, the reaction speed of phosphoramidite coupling can be at least 20 times slower, or at least 100 times slower, or at least 1000 times slower, or at least 10000 times slower, or at least 100000 times slower.

It is an object of the present disclosure to provide a good method for synthesizing a nucleic acid (analogue), which in some embodiments can be used for synthesizing a long nucleic acid, such as at least 100-mers or 1000-mers. It is a further object of the present disclosure to provide a good device associated therewith. This objective is accomplished by methods and devices described below.

Rather than growing a nucleic acid (analogue) strand sequentially through the cyclic addition of monomers—or even di- or trimers—using phosphoramidite chemistry, it was surprisingly realized that the above objective can be met by providing (e.g., synthesizing) nucleic acid fragments of a substantial length, for example, and then binding them together using phosphoramidite coupling. Indeed, assuming in a first approximation a constant stepwise coupling yield γ, the sequential addition of monomers to a length of n has a total yield of γ\hat{ }(n-1). For a length n of 100 and a stepwise coupling yield γ of 99%, this results in a total yield of only 37%; for a yield γ of 98% only 14%; etc. Conversely, the parallel synthesis—using the same sequential addition of monomers—of two strands of length n followed by binding both strands together has a total yield of γ\hat{ }(n-1)×δ\hat{ }, wherein δ is the yield of combining both strands (or more generally, for assembling m strands of length n—and assuming in first approximation also a constant combination yield δ—the total yield is γ\hat{ }(n-1)×δ\hat{ }(m-1)). Combining two strands of length 50 (to yield the same total length 100 as above), and even assuming a substantially lower yield δ of only 80%, for a yield γ of 99% this results in a total yield of 49%; for a yield γ of 98% still 30%; etc.

From the above, it will be clear that the length for the nucleic acid (analogue) fragments before coupling them together generally depends on several factors, including the yield of synthesizing the individual fragments and the yield of combining them. Notwithstanding, typically a worthwhile improvement (compared to sequentially synthesizing the entire nucleic acid (analogue)) can already be achieved for fragments with a length of at least 5 monomers; with the gain increasing for longer fragments. While it is still typically beneficial over sequentially synthesizing the entire nucleic acid (analogue), for increasingly long fragments not only the yield of synthesizing them but also the yield of combining them may drop off significantly. However, this can in turn be alleviated by applying the approach of the present disclosure multiple times (optionally in (semi-) parallel manner), e.g., in a sequential or hierarchical manner. For example, four fragments A, B, C, and D can be provided and combined either as: A+B→A−B, C+D→C−D, and A−B+C−D→A−B−C−D (i.e., hierarchically); or as: A+B→A−B, A−B+C→A−B−C, and A−B−C+D→A−B−C−D (i.e., sequentially).

Beyond the increase in practically achievable length (through increasing the total yield), this approach moreover allows for the different fragments to be synthesized in parallel (e.g., on different substrates, or on different areas of the same substrate), thereby also substantially decreasing the total time needed to synthesize the whole nucleic acid (analogue).

As a result, in some embodiments, the total yield of synthesizing the nucleic acid (analogue) can be increased. And in some embodiments, the length of the nucleic acid (analogue) which can be practically achieved can be increased.

In some embodiments of the present disclosure, the root fragment and the further fragment can be provided (e.g., synthesized) in parallel. In some embodiments of the present disclosure, the total time needed to synthesize the nucleic acid (analogue) can be decreased.

In some embodiments of the present disclosure, the approach disclosed herein can be applied multiple times, allowing for even longer nucleic acid fragments to be synthesized with maintaining relatively high yields.

In some embodiments of the present disclosure, the method can include inherent purification, thereby obviating a dedicated purification step.

In some embodiments of the present disclosure, the method can be adapted to accommodate different synthesis directions for the fragments.

In some embodiments of the present disclosure, inadvertent reactions between fragments can be reduced or prevented.

In some embodiments of the present disclosure, the nucleic acid (analogue) can be detached and collected for use in various applications.

In some embodiments of the present disclosure, the methods disclosed herein can be performed in a relatively straightforward and economical fashion.

In a first aspect, the present disclosure relates to a method for synthesizing a nucleic acid or nucleic acid analogue, comprising: a) providing a root fragment of the nucleic acid or nucleic acid analogue, b) providing a further fragment of the nucleic acid or nucleic acid analogue, c) phosphitylating either the root fragment or the further fragment, and d) binding the further fragment to the root fragment by a phosphoramidite coupling; wherein the root fragment and the further fragment each have a length of at least 5 monomers. In some embodiments, the root fragment and the further fragment can each have a length of at least 10 monomers. In some embodiments, the root fragment and the further fragment can each have a length of at least 20 monomers. In some embodiments, the root fragment and the further fragment can each have a length of at least 30 monomers. In some embodiments, the root fragment and the further fragment can each have a length of at least 50 monomers.

In some embodiments, a ratio of the length of the further fragment to the length of the root fragment (length further fragment: length root fragment) may be from 0.1 to 10, from 0.2 to 5, from 0.5 to 2, from 0.8 to 1.2, from 0.9 to 1.1, or from 0.95 to 1.05. The effect of the present disclosure on the efficiency (e.g., the total yield and/or speed) of the synthesis may typically be most pronounced if the root fragment and further fragment have a length which is at least in the same order of magnitude or substantially similar (e.g., having a ratio from 0.8 to 1.2). Notwithstanding, in some embodiments, the further fragment(s) may be more easily purified (e.g., to remove erroneous fragments) than the root fragment(s). Accordingly, in such embodiments it may nonetheless prove beneficial to start from a comparatively shorter (thereby having a lower error-rate) root fragment. For example, the ratio of the length of the further fragment to the length of the root fragment may be from 1.05 to 10, from 1.1 to 5, or from 1.2 to 2.

In some embodiments, the root fragment and the further fragment may each comprise at least two different monomers (e.g., two monomers having different nucleobases), at least three different monomers, or at least four different monomers.

In general, the root fragment and further fragment may be provided in step a and step b in any suitable way; for example, the root fragment and/or further fragment may be synthesized, separated from a mixture (e.g., from a biological sample), etc. Notwithstanding, step a may often comprise synthesizing the root fragment. Similarly, step b may often comprise synthesizing the further fragment. Synthesizing the root fragment and the further fragment may likewise be performed in any suitable way, such as by any suitable chemical, biochemical, and/or biological process. Notwithstanding, the root fragment and/or the further fragment may be synthesized using phosphoramidite synthesis. In some embodiments, the root fragment and/or the further fragment may be synthesized using a method comprising capping an erroneous fragment to prevent further chain elongation. Phosphoramidite synthesis is a well-established process for synthesizing new (i.e., not limited to copying existing strands) nucleic acids (analogues). Moreover, it relatively straightforwardly allows to cap erroneous (e.g., unreacted) fragments to prevent further chain elongation. In some embodiments where the root fragment and/or further fragment is to be attached to a substrate via a linker (cf. infra), the substrate may be pre-functionalized with the linker (i.e., prior to attaching the fragment and/or starting the fragment synthesis), or the linker may be attached to the substrate together with the fragment or as part of the fragment synthesis.

In some embodiments, the root fragment and the further fragment may be synthesized concurrently (i.e., simultaneously, in parallel). In some embodiments, the root fragment and/or the further fragment may be synthesized on a substrate. In some embodiments, the root fragment and the further fragment may be synthesized on a different substrate (or on separate areas of the same substrate). Alternatively, in some embodiments where the further fragment can be detached from the substrate selectively with respect to the root fragment, the root fragment and the further fragment may be synthesized on the same substrate (e.g., in the same area). Detaching the further fragment from the substrate selectively with respect to the root fragment may be performed in a way that is controlled by light, temperature, or (electro) chemistry, for example, by using a reversible bond or a cleavable linker (cf. infra). Selectively releasing the further fragment may be performed on a fragment-by-fragment (i.e., individual control of each fragment release) or collective-by-collective (e.g., having different areas with a plurality of fragments in each and releasing all of the fragments in an area simultaneously).

Step c is typically performed to obtain a phosphoramidite. In some embodiments, in step c the root fragment or further fragment may be phosphitylated while attached to a substrate. In some embodiments, step c may be performed in a same location (e.g., a same reaction chamber) as step d (i.e., in situ). For example, the root fragment may be phosphitylated while attached to the assembly substrate (cf. infra).

Compared to doing so in solution, phosphitylating the root fragment or further fragment while attached to a substrate contributes to preventing any inadvertent reaction between an already phosphitylated fragment and a yet to be phosphitylated fragment. In some embodiments, the root fragment or further fragment may be phosphitylated using a phosphitylating agent, such as chloro N,N,N′,N′-tetraisopropyl phosphorodiamidite. In some embodiments, the root fragment or further fragment may be phosphitylated using the phosphitylating agent in presence of a base and an activator (e.g., a triazole activator).

Typically, in step d the further fragment may be dispersed (e.g., dissolved or suspended) in a reaction medium (e.g., a solvent or buffer). This allows for easily contacting and reacting the further fragment with the root fragment. In some embodiments, in step d the root fragment may be attached to a substrate (e.g., an assembly substrate). This allows for immobilizing the root fragment—and thus also the nucleic acid (analogue) resulting from step d—and simultaneously protecting the end of the root fragment which is not intended to be coupled with the further fragment (cf. infra). In other embodiments, in step d the root fragment and the further fragment may both be dispersed in the reaction medium.

The root fragment and the further fragment generally each have two ends, typically referred to as 3′ end (i.e., 3′ carbon of the pentose sugar at one extreme of the fragment) and 5′ end (i.e., 5′ carbon of the pentose sugar at the opposite extreme of the fragment). The 3′ end is a secondary carbon while 5′ end is a primary carbon, as such 5′ end is typically more reactive due to steric hindrance and a lower stabilizing effect through the attached groups. Normally, the desired binding of the further fragment to the root fragment is moreover by coupling 3′ end of one fragment to 5′ end of the other fragment (i.e., not 3′ to 3′ or 5′ to 5′, as this would yield a nucleic acid (analogue) with a distorted directionality).

In some embodiments therefore, the desired directionality of the binding of the root fragment and further fragment may be realized by using the inherent difference in reaction kinetics between 3′ end and 5′ end. In some embodiments, the root fragment may have its 5′ end protected (i.e., rendered less reactive or unreactive to phosphoramidite coupling, e.g., through being attached through the end to the assembly substrate). Meanwhile the further fragment may have both ends reactive (i.e., not protected). Accordingly, with 3′ end of the root fragment phosphitylated, the more reactive 5′ end of the further fragment will more readily bind through phosphoramidite coupling to the root fragment, thereby realizing the desired directionality. In some embodiments, it may be less preferred to phosphitylate the further fragment—as opposed to the root fragment—, as—given that both ends of the further fragments are reactive—this could lead to inadvertent reactions between already phosphitylated fragments and yet to be phosphitylated fragments, thereby reducing the overall yield of the desired nucleic acid (analogue).

Alternatively, in some embodiments, in step d the root fragment and the further fragment may each have an end protected (i.e., rendered less reactive or unreactive to phosphoramidite coupling). For the root fragment, this may for example involve being attached through the end to the assembly substrate. For the further fragment—and as an alternative to the substrate for the root fragment—, this may involve the end being functionalized with a protective group. To that end, in some embodiments, the root fragment and/or the further fragment may prior to step d (e.g., during their synthesis) be at one end attached to the substrate by a linker, the linker being such that the fragment can be detached from the substrate while leaving a protective group on the end rendering the end less reactive or unreactive to phosphoramidite coupling. For example, the fragment may be through the to-be-protected end attached either to a linker which is in turn attached to the substrate by a reversible bond, or to a cleavable linker. The reversible bond may be for instance be a thermo-, pH-, redox-, and/or photolabile bond, or, similarly, the cleavable linker may be a thermo-, pH-, redox-, and/or photolabile linker. Accordingly, upon detaching or cleaving the linker, at least a portion of the linker remains attached to and protects the end of the fragment. Alternatively, the end may be protected by directly functionalizing the end with the protective group (e.g., while the fragment is attached to a substrate through the opposite end).

Regarding the directionality of the fragments in certain embodiments, to realize a 3′ to 5′ (or 5′ to 3′) binding of the further fragment to the root fragment, the root fragment and further fragment should generally be protected at opposite ends. For example, where the root fragment is protected through its attachment to the assembly substrate, the root fragment may be attached to the assembly substrate through its 5′ end and the further fragment may comprise a protective group at its 3′ end, or the root fragment may be attached to the assembly substrate through its 3′ end and the further fragment may comprise a protective group at its 5′ end. Accordingly, the synthesis direction of the further fragment may also typically depend on the orientation of the root fragment and the way the protection of the further fragment is realized: if the protected end is the end through which the further fragment was attached to the synthesis substrate, the synthesis direction (e.g., 3′ to 5′) of the further fragment is typically opposite to the orientation (e.g., 5′ to 3′, 5′ end being attached to the assembly substrate) of the root fragment (and thus, if the root fragment is synthesized on the assembly substrate, the synthesis direction of the root fragment and further fragment may typically be opposite). More specifically, the further fragment may in such a case be synthesized on a synthesis substrate: in a 3′ to 5′ direction if the root fragment is attached to the assembly substrate through its 5′ end, or in a 5′ to 3′ direction if the root fragment is attached to the assembly substrate through its 3′ end. Conversely, if the protected end is the end away from the substrate, the synthesis direction of the further fragment is typically equal to the orientation of the root fragment.

In some embodiments wherein the root fragment and/or the further fragment are synthesized using a method comprising capping an erroneous fragment to prevent further chain elongation (cf. supra), providing the root fragment and the further fragment with the end rendered less reactive or to phosphoramidite coupling may be performed such that erroneous fragments are less reactive or unreactive to phosphoramidite coupling at both ends. Since such capped and/or protected fragments are not readily available for binding with another fragment, an inherent purification is achieved without needing a dedicated purification step.

In some embodiments where the root fragment is attached to the assembly substrate, the method may further comprise after step d detaching the nucleic acid (analogue) from the assembly substrate. This can for instance be realized by the root fragment being attached i) directly to the assembly substrate or to a linker on the assembly substrate by a reversible bond, or ii) to a linker attached to the assembly substrate by a reversible bond or to a cleavable linker. As previously described, the reversible bond may be a thermo-, pH-, redox-, and/or photolabile bond, or, similarly, the cleavable linker may be a thermo-, pH-, redox-, and/or photolabile linker. Detaching the nucleic acid (analogue) allows for collecting it and using elsewhere (in the same device or in another location) for its intended application.

In some embodiments where the root fragment and/or further fragment is protected at one end by a protective group, the method may further comprise after step d removing the protective group. Removing the protective group allows for recovering the reactive end, which may be needed or useful for the synthesized nucleic acid (analogue)'s intended application; including using it as a new root fragment or further fragment (cf. infra). Note that where the protective group was provided as at least a portion of a linker remaining attached to the fragment after detaching or cleaving the linker, removing the protective group typically entails that the linker was detachable and/or cleavable at two locations, each with its own distinct trigger (e.g., one being thermolabile and the other photolabile, or both being photolabile but at different wavelengths).

In some embodiments, after step d the root fragment extended with the further fragment may become a new root fragment or a new further fragment. In some embodiments, steps b to d may be repeated at least 1 time; at least 2 times at least 5 times, at least 10 times, or at least 20 times.

In some embodiments, any feature of any embodiment of the first aspect may independently be as correspondingly described for any embodiment of any of the other aspects.

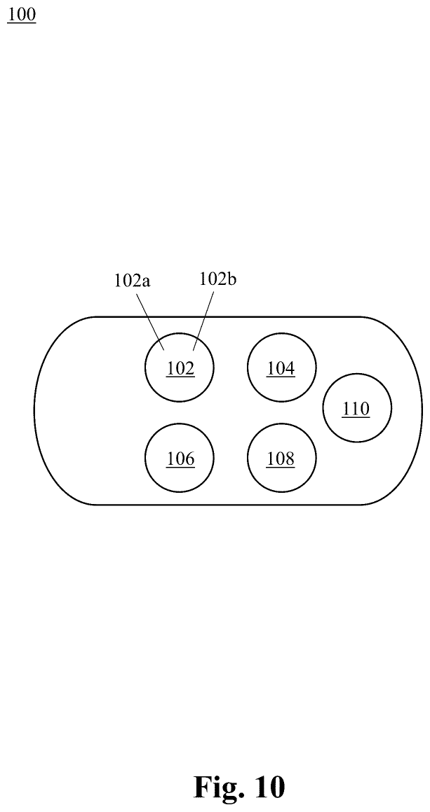

In a second aspect, as shown in FIG. 10, the present disclosure relates to a device 100 for synthesizing a nucleic acid or nucleic acid analogue using the method according to any embodiment of the first aspect. In some embodiments, the device 100 can include i) a component 102 for receiving and/or synthesizing the root fragment and/or the further fragment of the nucleic acid or nucleic acid analogue, ii) a component 104 for phosphitylating either the root fragment or the further fragment, and iii) a component 106 for binding the further fragment to the root fragment by a phosphoramidite coupling. The component 102 can further include an assembly substrate (not shown) and synthesizing substrate (not shown).

In some embodiments, component i 102 may be for synthesizing the root fragment, the further fragment, or both. In some embodiments, component i 102 may be for synthesizing the root fragment, and the device may further comprise: iv) a component 108 for synthesizing the further fragment.

In some embodiments, the device may further comprise: iv) a component 110 for detaching the synthesized nucleic acid (analogue) from an assembly substrate (not shown).

In some embodiments, one or more of the components may be a single component (i.e., they need not be distinct). For example, components i, ii, iii and—if present—iv may be a single component (e.g., a reaction chamber) in which the root fragment (or further fragment) is provided (e.g., synthesized), phosphitylated, bound to the further fragment (or root fragment) and optionally the resulting nucleic acid (analogue) is detached.

In some embodiments, the component i 102 for synthesizing the root fragment and/or the further fragment may comprise a plurality (i.e., 2 or more) of synthesis sites 102a, 102b (or areas; cf. supra) wherein each synthesis site is configured for synthesizing at least one root fragment or at least one further fragment (and can contain an assembly substrate and/or a synthesis substrate). In some embodiments, each synthesis site may be configured for synthesizing a plurality of root fragments or further fragments. Advantageously, the simultaneous synthesis of the same type of (e.g., sequentially identical) root fragments or further fragments can thereby be multiplexed.

In some embodiments, the component i 102 for synthesizing the root fragment and/or the further fragment may comprise a plurality (i.e., 2 or more) of synthesis sites 102a, 102b (or areas; cf. supra) wherein at least two different types of (e.g., being sequentially different) root fragments are synthesized on two different synthesis sites simultaneously, and/or at least two different types of further fragments are synthesized on two different synthesis sites simultaneously. In some embodiments, the simultaneous synthesis of different types of (e.g., being sequentially different) root fragments and/or further fragments can be multiplexed. Each of the synthesis sites can be configured to be addressed individually by the device (e.g., being activated individually by light, temperature and/or (electro) chemistry).

In some embodiments, at least one synthesis site may comprise an electrode (not shown) configured to electrochemically synthesize the root fragment or further fragment. In some embodiments, each synthesis site may comprise such an electrode.

In some embodiments, any feature of any embodiment of the second aspect may independently be as correspondingly described for any embodiment of any of the other aspects.

The present disclosure will now be described by a detailed description of several embodiments. Other embodiments of the disclosure can be configured according to the knowledge of the person skilled in the art without departing from the true technical teachings of the disclosure.

### Example 1

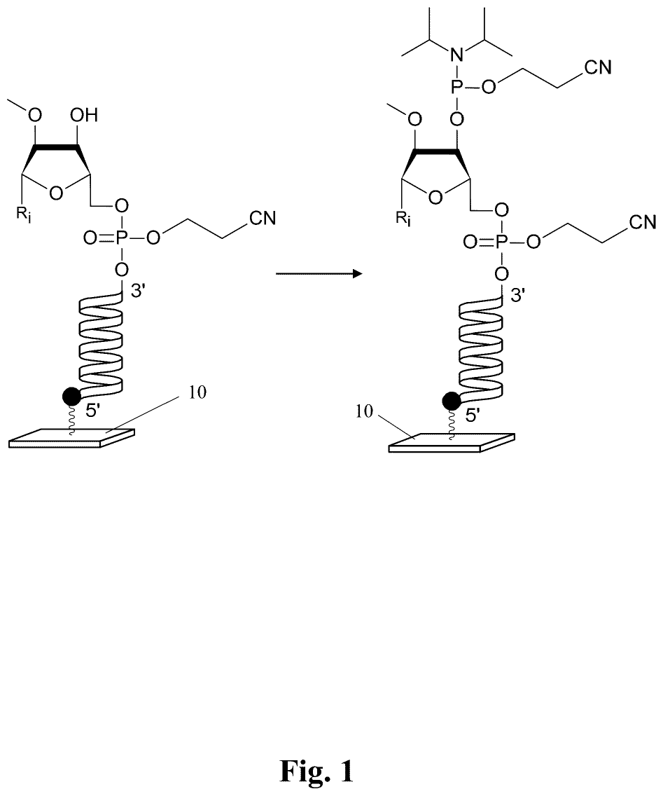

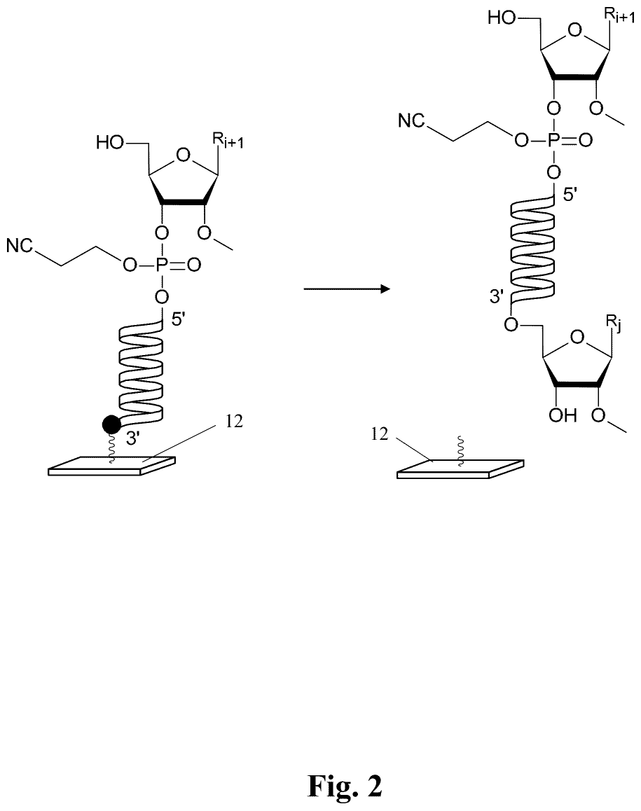

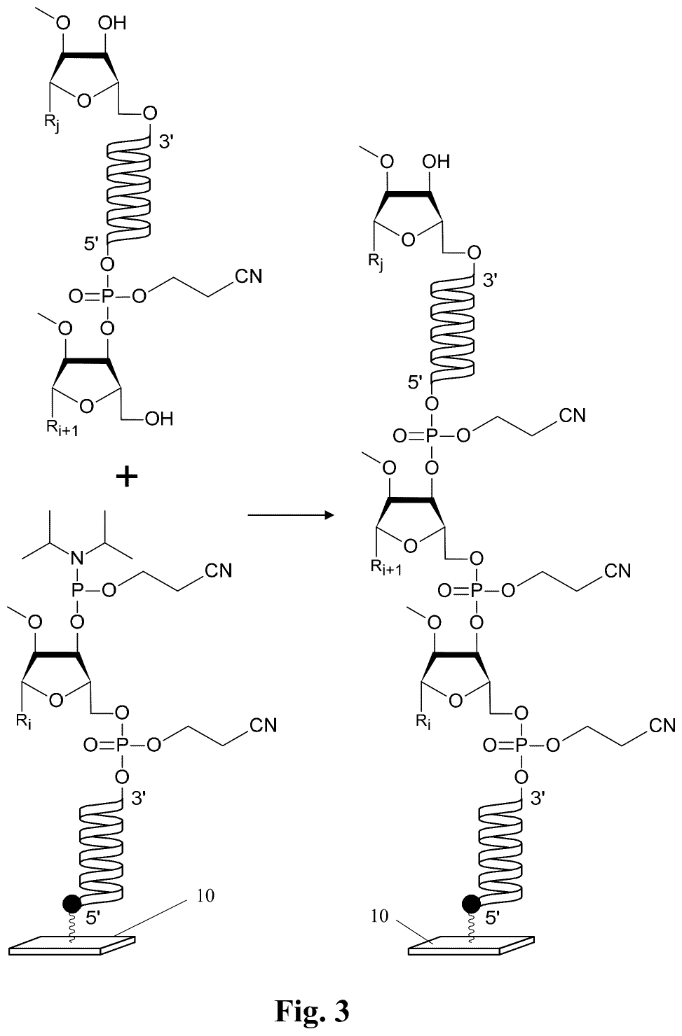

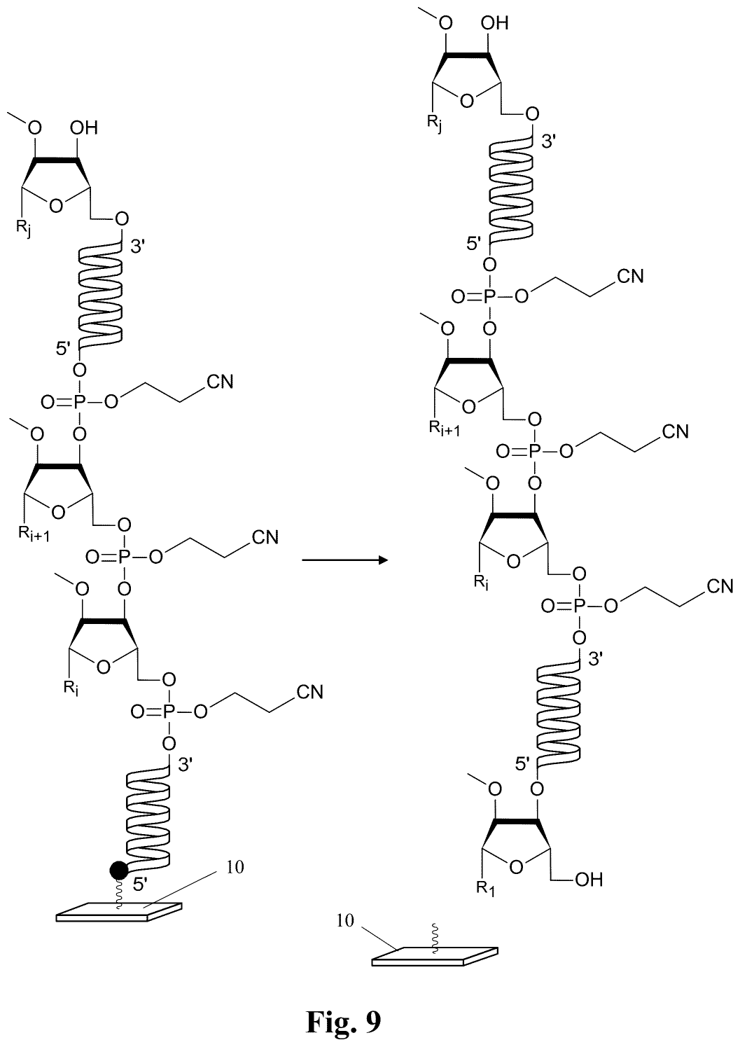

A first example of a nucleic acid (analogue) synthesis in accordance with the present disclosure is schematically depicted in FIG. 1, FIG. 2, FIG. 3, and FIG. 9.

This example starts with the provision of the root fragment (FIG. 1, left side); here attached to an assembly substrate 10. This root fragment can for instance be obtained by attaching a starting fragment (e.g., a nucleoside or (oligo) nucleotide) to the assembly substrate 10 and elongating it in situ by sequentially adding (e.g., using phosphoramidite coupling) nucleosides or (oligo) nucleotides. Alternatively or complementarily, the root fragment may be (at least partially) formed elsewhere and subsequently attached to the assembly substrate 10.

Next, the root fragment is phosphitylated while attached to the assembly substrate 10 (FIG. 1).

In parallel, a further fragment is provided (FIG. 2). Similar to the root fragment, the further fragment can for instance be obtained by attaching a starting fragment (e.g., a nucleoside or (oligo) nucleotide) to a synthesis substrate 12 and elongating it by sequentially adding (e.g., using phosphoramidite coupling) nucleosides or (oligo) nucleotides. Subsequently, the further fragment is detached from the synthesis substrate 12. This can for instance be achieved through the further fragment being attached directly to the synthesis substrate 12 or to a linker on the synthesis substrate 12 by a reversible bond, such as a thermo-, pH-, and/or photolabile bond.

After being detached from the synthesis substrate 12, the further fragment is brought near the phosphitylated root fragment and allowed to react in a phosphoramidite coupling, thereby binding the further fragment to the root fragment (FIG. 3).

Optionally, the attachment of the root fragment to the assembly substrate 10 can be reversible, so that the synthesized nucleic acid (analogue) can be detached from the assembly substrate 10 (FIG. 9). As desired, e.g., in view for the further use of the nucleic acid (analogue), this may involve the root fragment being attached either i) directly to the assembly substrate 10 or to a linker on the assembly substrate 10 by a reversible bond or ii) to a linker attached to the assembly substrate 10 by a reversible bond or to a cleavable linker. Here, the reversible bond may again be a thermo-, pH-, redox-, and/or photolabile bond; or, similarly, the cleavable linker may be a thermo-, pH-, redox-, and/or photolabile linker.

After the above synthesis, the obtained nucleic acid (analogue) may be used in a variety of applications, including being used as a root fragment or further fragment in a new synthesis in accordance with the present disclosure.

Note that as depicted, the root fragment is attached to the assembly substrate 10 via its 5′ end; entailing that if the root fragment is (at least partially) formed in situ, this involves a reverse (i.e., 5′ to 3′) synthesis. Although not strictly necessary, this is one preferred orientation for the root fragment in the present example, where both 3′ and 5′ end of the further fragment are in principle free to react. Indeed, knowing that the 5′ end of the further fragment is typically more reactive than its 3′ end (cf. supra), this orientation sets up the root fragment to couple with the further fragment in the desired 3′ (of the root fragment) to 5′ (of the further fragment) coupling. If both ends are indeed “free” (i.e., are reactive, e.g., in the form of bearing-OH group), the synthesis orientation of the further fragment can be selected as desired. Notwithstanding, if providing the further fragment comprises capping erroneous (e.g., unreacted) strands to prevent them from growing further, as is commonly done in phosphoramidite synthesis, it may be preferred to synthesize the further fragment in the forward (i.e., 3′ to 5′) direction. This will result in the erroneous strands being capped off at the more reactive 5′ end, thereby deterring these erroneous strands from coupling with a root fragment more than if the less reactive 3′ was capped off.

### Example 2

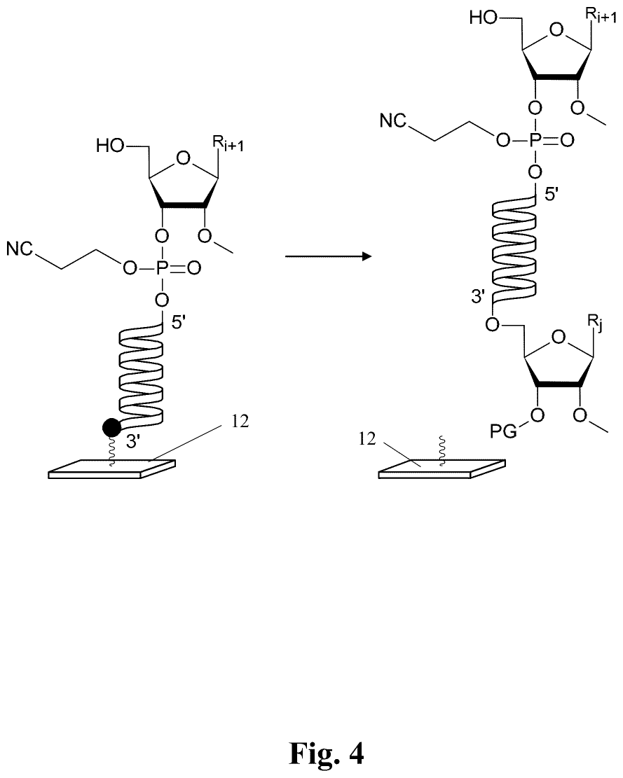

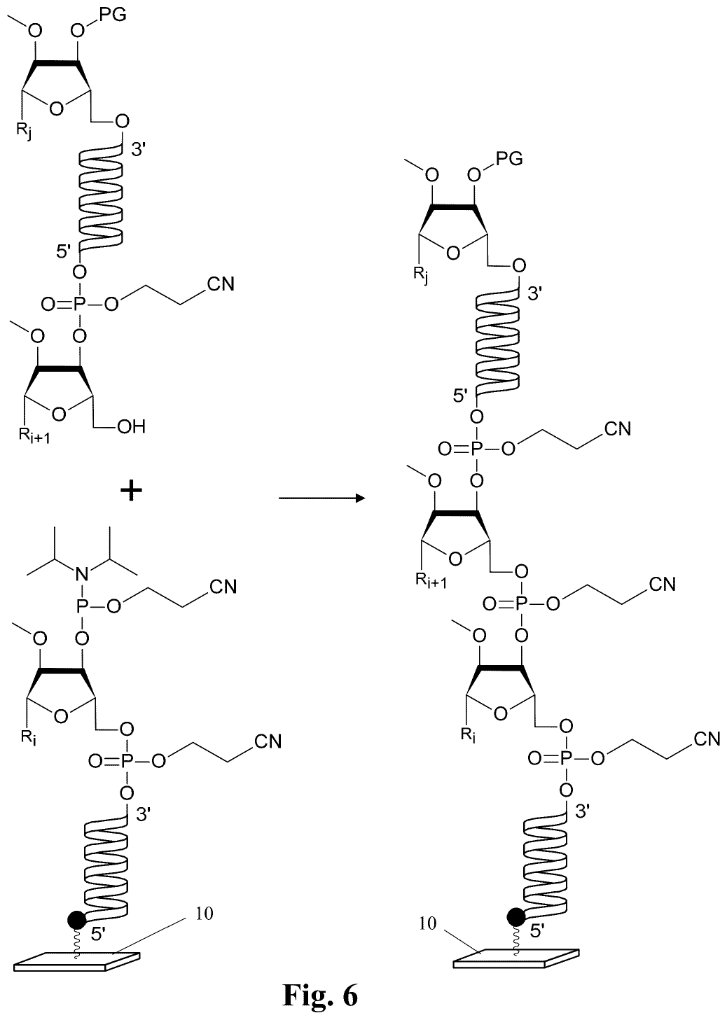

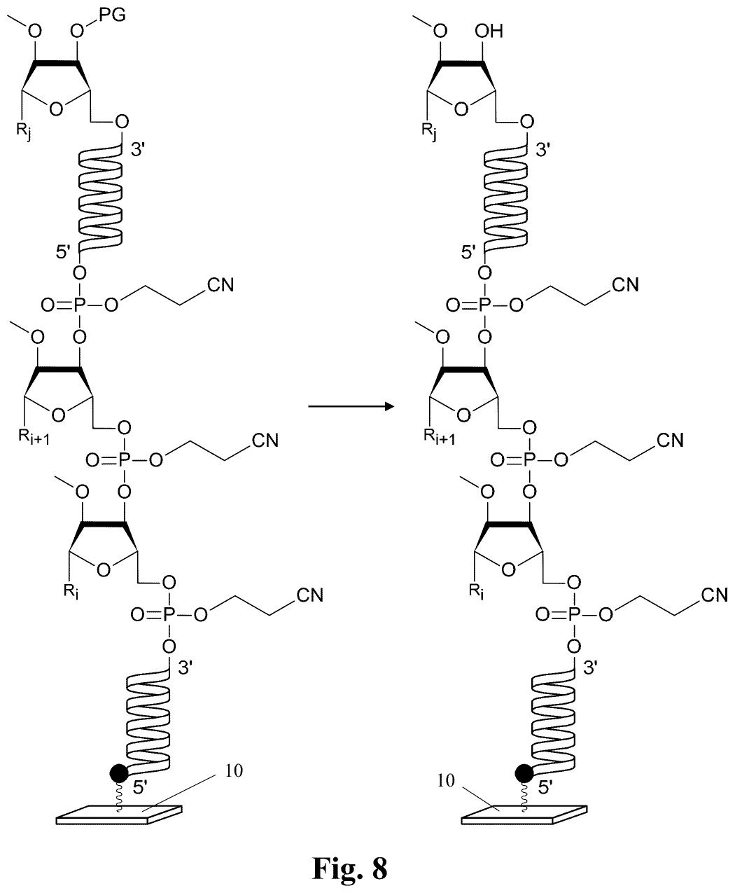

A second example of a nucleic acid or nucleic acid (analogue) synthesis in accordance with the present disclosure is schematically depicted in FIG. 1, FIG. 4, FIG. 6, FIG. 8 and FIG. 9.

This example starts the same as Example 1, with the provision of the root fragment attached to the assembly substrate 10 and subsequent phosphitylation of the root fragment (FIG. 1).

Also, the provision of the further fragment is similar to that of Example 1 but with the difference that after detaching the further fragment from the synthesis substrate 12, one end (3′ or 5′) is protected by bearing a protective group (PG)—rendering the end less reactive (or unreactive) to phosphoramidite coupling (compared to the end bearing an —OH group)—while the other end remains unaltered (e.g., as a reactive end). As depicted in FIG. 4, this can be realized by attaching the further fragment to the synthesis substrate 12 in such a way that after detaching the further fragment the protective group is formed (or remains) at the end through which the further fragment was attached to the synthesis substrate 12. For instance, the further fragment may be attached to the synthesis substrate 12 by a cleavable or detachable linker, such that at least a portion of the linker remains attached to the further fragment upon cleaving/detaching the linker. Alternatively, the protection at one end of the further fragment may also be provided simply by functionalizing said end with the protective group. Preferably, this can be done while the further fragment is still attached to the synthesis substrate (not depicted), thereby ensuring that only one end is protected (and that this protected end is the same for each further fragment). In this case, the protected end is then the end away from the substrate (i.e., opposite to what is depicted in FIG. 4) and the other end is freed by detaching the further fragment as in Example 1.

Next, the detached further fragment is again brought near the phosphitylated root fragment and allowed to react in a phosphoramidite coupling, thereby binding the further fragment to the root fragment (FIG. 6).

Preferably, the protection of one end of the further fragment is removable. FIG. 8, for example, shows the protective group being removed, thereby recovering a reactive end, after coupling the further fragment to the root fragment, while the resulting nucleic acid (analogue) is still attached to the assembly substrate 10. Notwithstanding, it will be clear that the protection could also be removed after detaching the nucleic acid (analogue). Removing the protection is particularly useful when the protected end should be rendered reactive again; for instance, because it is to participate in a new synthesis in accordance with the present disclosure.

Finally, the attachment of the root fragment to the assembly substrate 10 is optionally reversible, so that the synthesized nucleic acid (analogue) can be detached from the assembly substrate 10 (FIG. 9).

Note that as depicted, the root fragment is also attached to the assembly substrate 10 via its 5′ end. However, since (in contrast to Example 1) the coupling between the root fragment and the further fragment does not rely on a difference in reaction kinetics between a free 3′ and a free 5′ end, the opposite orientation is equally possible in the present example. Depending on the orientation of the root fragment and the way the protection is realized (i.e., at the end through which the further fragment was attached to the synthesis substrate 12 or at the end away from the substrate), the further fragment can be synthesized in the forward or reverse direction. Generally though, because the further fragment and root fragment are typically to be coupled in a 3′ to 5′ (or 5′ to 3′) fashion, if the protected end is the end through which the further fragment was attached to the synthesis substrate 12, the synthesis direction (e.g., 3′ to 5′) of the further fragment is typically opposite to the orientation (e.g., 5′ to 3′, 5′ end being attached to the assembly substrate 10) of the root fragment; and vice versa: if the protected end is the end away from the substrate 10, the synthesis direction of the further fragment is typically equal to the orientation of the root fragment. The above notwithstanding, if providing the further fragment comprises capping erroneous strands to prevent them from growing further, it may be preferred to protect the end through which the further fragment was attached to the synthesis substrate 12, and thus to synthesize the further fragment in the direction opposite of the orientation of the root fragment. This will result in the erroneous strands being capped off at the non-protected end, thereby rendering these erroneous strands unreactive at both ends and thus preventing them from coupling with a root fragment.

### Example 3

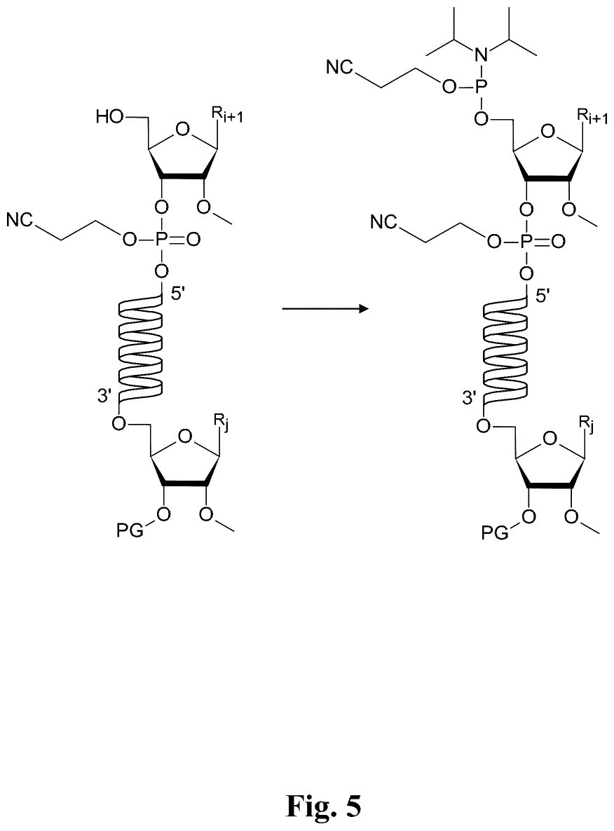

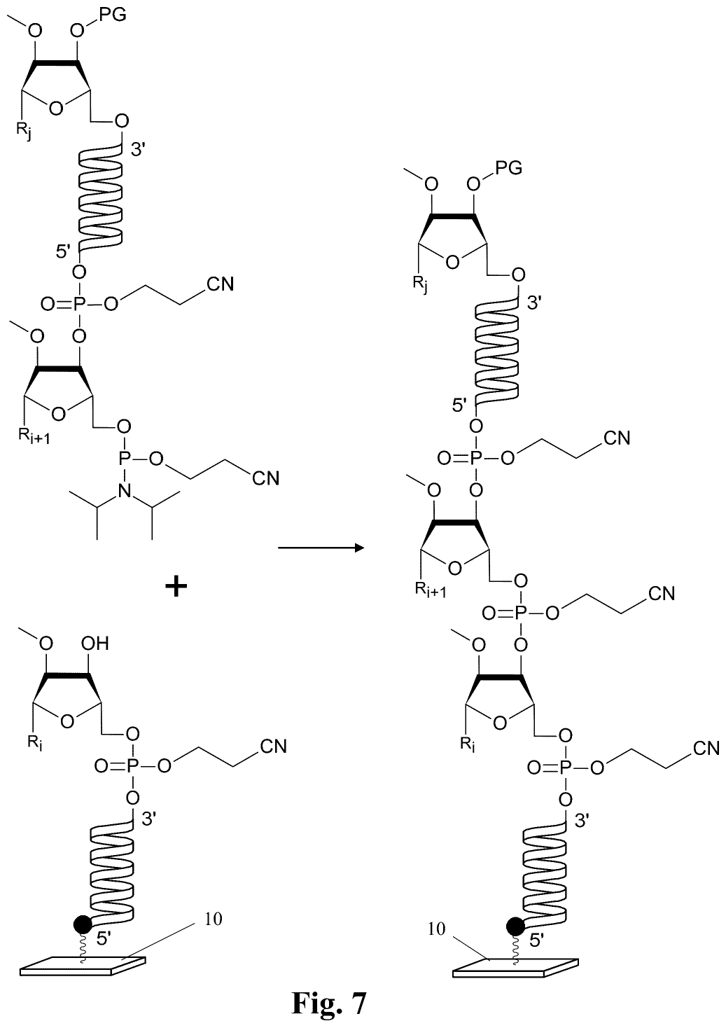

A third example of a nucleic acid or nucleic acid (analogue) synthesis in accordance with the present disclosure is schematically depicted in FIG. 4, FIG. 5, FIG. 7, FIG. 8 and FIG. 9.

This example is similar to Example 2, but with the difference that not the root fragment but the further fragment is phosphitylated prior to coupling both. Accordingly, the root fragment is provided and left as is (FIG. 1, left side), and the further fragment is provided and outfitted with a protective group at one end (e.g., FIG. 4); both as previously described.

Next, the further fragment is phosphitylated. As depicted in FIG. 5, this is performed after the further fragment was detached from the synthesis substrate 12; however, where the protected end is the end through which the further fragment was attached to the synthesis substrate 12, the phosphitylation may already be performed while the further fragment is still attached to the synthesis substrate 12. The latter may even be preferred in that case, as phosphitylating the further fragments in solution, depending on the speed of completion, risks that a yet to be phosphitylated further fragment would inadvertently couple (in a 3′ to 3′ or 5′ to 5′ fashion) with an already phosphitylated further fragment; which risk can be (at least partially) mitigated by performing the phosphitylation while the further fragments are still attached to the substrate 12.

Subsequently, the phosphitylated further fragment is brought near the root fragment and allowed to react in a phosphoramidite coupling, thereby again binding the further fragment to the root fragment (FIG. 7).

Preferably, the protection of one end of the further fragment is again removable; which may be performed either while the resulting nucleic acid (analogue) is still attached to the assembly substrate 10 (as shown in FIG. 8) or after detaching the nucleic acid (analogue).

Finally, the attachment of the root fragment to the assembly substrate 10 is optionally also reversible, so that the synthesized nucleic acid (analogue) can be detached from the assembly substrate 10 (FIG. 9).

Note that as depicted, the root fragment is again attached to the assembly substrate 10 via its 5′ end. However, the opposite orientation is also equally possible in this example, since once more (in contrast to Example 1) the coupling between the root fragment and the further fragment does also not rely on a difference in reaction kinetics between a free 3′ and a free 5′ end. Depending on the orientation of the root fragment and the end where the protection is realized, the further fragment can again be synthesized in the forward or reverse direction. Again, generally though, because the further fragment and root fragment are typically to be coupled in a 3′ to 5′ (or 5′ to 3′) fashion, if the protected end is the end through which the further fragment was attached to the synthesis substrate 12, the synthesis direction (e.g., 3′ to 5′) of the further fragment is typically opposite to the orientation (e.g., 5′ to 3′, 5′ end being attached to the assembly substrate 10) of the root fragment, and vice versa. The above notwithstanding, if providing the further fragment comprises capping erroneous strands to prevent them from growing further, it may again be preferred to protect the end through which the further fragment was attached to the synthesis substrate 12, and thus to synthesize the further fragment in the direction opposite the orientation of the root fragment. This will result in the erroneous strands being capped off at the non-protected end, thereby rendering these erroneous strands unreactive at both ends and thus preventing them from coupling with a root fragment.

It is to be understood that although preferred embodiments, specific constructions, configurations and materials have been discussed herein in order to illustrate the present disclosure. It will be apparent to those skilled in the art that various changes or modifications in form and detail may be made without departing from the scope of the disclosure as defined in the appended claims.

Features from the dependent claims may be combined with features of the independent claims and with features of other dependent claims as appropriate and not merely as explicitly set out in the claims.

What is claimed is:

1. A method for synthesizing a nucleic acid or nucleic acid analogue, comprising:
a) providing a root fragment of the nucleic acid or nucleic acid analogue;
b) providing a further fragment of the nucleic acid or nucleic acid analogue;
c) phosphitylating either the root fragment or the further fragment; and
d) binding the further fragment to the root fragment by a phosphoramidite coupling,
wherein the root fragment and the further fragment each have a length of at least 5 monomers.

2. The method of claim 1, wherein a ratio of the length of the further fragment to the length of the root fragment is from 0.8 to 1.2.

3. The method of claim 1, wherein in step d the root fragment and the further fragment each have an end rendered unreactive to phosphoramidite coupling.

4. The method of claim 1, wherein in step d the root fragment is attached to a substrate.

5. The method of claim 1, wherein the root fragment and/or the further fragment are synthesized on a substrate.

6. The method of claim 5, wherein the root fragment and/or the further fragment is attached at one end to the substrate by a linker, the linker being such that the fragment can be detached from the substrate while leaving a protective group on the end rendering the end unreactive to phosphoramidite coupling.

7. The method of claim 1, wherein the root fragment and/or the further fragment are synthesized using a method comprising capping an erroneous fragment to prevent further chain elongation, and wherein providing the root fragment and the further fragment with the end rendered unreactive to phosphoramidite coupling is performed such that erroneous fragments are unreactive to phosphoramidite coupling at both ends.

8. The method of claim 7, wherein the root fragment and/or the further fragment are synthesized using phosphoramidite synthesis.

9. The method of claim 1, wherein in step c the root fragment or further fragment is phosphitylated while attached to a substrate.

10. The method of claim 1, wherein the root fragment or further fragment is phosphitylated using chloro N,N,N′,N′-tetraisopropyl phosphorodiamidite.

11. The method of claim 1, wherein after step d the root fragment extended with the further fragment becomes a new root fragment or a new further fragment, and wherein steps a to d are repeated at least 1 time.

12. The method of claim 1, wherein the root fragment and the further fragment each have a 5′ and a 3′ end, and wherein in step d
the root fragment is attached to an assembly substrate through its 5′ end and the further fragment comprises a protective group at its 3′ end, or
the root fragment is attached to an assembly substrate through its 3′ end and the further fragment comprises a protective group at its 5′ end.

13. The method according to claim 12, wherein the root fragment is phosphitylated while attached to the assembly substrate.

14. The method of claim 12, wherein the further fragment is synthesized on a synthesis substrate in a 3′ to 5′ direction if the root fragment is in step d attached to the assembly substrate through its 5′ end or in a 5′ to 3′ direction if the root fragment is in step d attached to the assembly substrate through its 3′ end.

15. A device for synthesizing a nucleic acid or nucleic acid analogue using the method of claim 1, comprising:
a) a component for receiving the root fragment and the further fragment of the nucleic acid or nucleic acid analogue;
b) a component for phosphitylating either the root fragment or the further fragment; and
c) a component for binding the further fragment to the root fragment by a phosphoramidite coupling.
